(19) United States
(12) Patent Application Publication
(10) Pub. No.: US 2009/0048288 A1
(43) Pub. Date: Feb. 19, 2009

(54) METHOD OF TREATING STRESS-MEDIATED DEPRESSION

(75) Inventors: Bjarke Ebert, Farum (DK); Torsten Meldgaard Madsen, Charlottenlund (DK)

Correspondence Address: DARBY & DARBY P.C., P.O. BOX 770, Church Street Station, New York, NY, 10008-0770, US

(73) Assignee: H. Lundbeck A/S, Valby (DK)

(21) Appl. No.: 12/187,865

(22) Filed: Aug. 7, 2008

Related U.S. Application Data
(60) Provisional application No. 60/955,463, filed on Aug. 13, 2007.

(51) Int. Cl. A61K 31/437; C12Q 1/28; C12Q 1/52; C12Q 1/37; G01N 33/68; A61P 25/00; A61P 25/24

(52) U.S. Cl. 514/302; 435/28; 436/86; 435/16; 435/23

(57) ABSTRACT

The present invention relates to a method for the treatment of depression comprising administering a therapeutically effective amount of gaboxadol to a patient, wherein the level of one or more inflammatory markers in said patient is increased or abnormal. The present invention also relates to a method for the treatment of stress-mediated depression comprising administering a therapeutically effective amount of gaboxadol to a patient, wherein the level of one or more inflammatory markers is increased or abnormal in said patient. The present invention also relates to a method for the treatment of depression or the amelioration of one or more depressive symptoms comprising administering a therapeutically effective amount of gaboxadol to a patient, wherein the clinical presentation of one or more symptoms of depression are the physiological effect of a general medical condition. Furthermore the present also relates to a method for testing the therapeutic effectiveness of a compound in the treatment of depression or reducing the symptoms of depression comprising measuring the amount of one or more inflammatory markers in a sample from a patient before said compound is administered to the patient and comparing with the amount of said one or more inflammatory markers in a sample from the same patient after administration of said compound to the patient.

This application claims the benefit of U.S. Provisional Application No. 60/955,463, filed Aug. 13, 2007, which is hereby incorporated by reference.

### FIELD OF THE INVENTION

The present invention relates to a method for the treatment of depression comprising administering a therapeutically effective amount of gaboxadol to a patient, wherein the level of one or more inflammatory markers in said patient is increased or abnormal. The present invention also relates to a method for the treatment of stress-mediated depression comprising administering a therapeutically effective amount of gaboxadol to a patient, wherein the level of one or more inflammatory markers is increased or abnormal in said patient. The present invention also relates to a method for the treatment of depression or the amelioration of one or more depressive symptoms comprising administering a therapeutically effective amount of gaboxadol to a patient, wherein the clinical presentation of one or more symptoms of depression are the physiological effect of a general medical condition. Furthermore the present also relates to a method for testing the therapeutic effectiveness of a compound in the treatment of depression or reducing the symptoms of depression comprising measuring the amount of one or more inflammatory markers in a sample from a patient before said compound is administered to the patient and comparing with the amount of said one or more inflammatory markers in a sample from the same patient after administration of said compound to the patient.

### BACKGROUND OF THE INVENTION

Selective serotonin reuptake inhibitors (hereinafter referred to as SSRIs) have become first choice therapeutics in the treatment of depression, certain forms of anxiety and social phobias, because they are effective, well tolerated and have a favourable safety profile compared to the classic tricyclic antidepressants.

However, clinical studies on depression and anxiety disorders indicate that non-response to SSRIs is substantial, up to 30%. Another, often neglected, factor in antidepressant treatment is compliance, which has a rather profound effect on the patient's motivation to continue pharmacotherapy.

Increasing evidence suggest that there exists a link between inflammation and depression. It has for example been found that some depressed patients have higher levels of inflammatory markers, such as proinflammatory cytokines, acute phase proteins, chemokines and cellular adhesion molecules. Moreover it has been found that therapeutic administration of the cytokine interferon-α leads to depression in up to 50% of patients. Stress, such as past emotional experiences, which can precipitate depression, can also promote inflammatory responses through effects on sympathetic and parasympathetic nervous system pathways (Raison et al., 2006, Trends in Immunology, vol 27, no. 1, 24-31). Additionally, physiological stress as the result of a medical illness or by coping with the illness or the medication associated with it, in many cases leads to a depressive disorder or depressive symptoms. Physiological stress is also linked to higher levels of inflammatory markers, such as proinflammatory cytokines, acute phase proteins, chemokines and cellular adhesion molecules.

Taken together the above suggests that reversal of increased inflammatory markers might treat depression, especially stress-mediated depression or the stress-mediated depressive symptoms in patients having increased inflammatory markers.

Gaboxadol (4,5,6,7-tetrahydroisoxazolo[5,4-c]pyridine-3-ol) (THIP), a selective extrasynatic GABA_{A} agonist, is described in EP Patent No. 0000338 and in EP Patent No. 0840601, and has previously shown great potential in the treatment of sleep disorders. Gaboxadol has the following general formula:

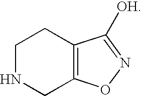

Gaboxadol may be prepared using methods that are well known in the art. For example as disclosed in EP Patent No. 0000338.

WO2004112786 discloses the use of gaboxadol for the treatment of depression as monotherapy or as combination therapy with other drugs, in particular that gaboxadol may be combined with a serotonin reuptake inhibitor, such as for example escitalopram for the treatment of depression. This disclosure thus broadly describes the use of gaboxadol for the treatment of depression as monotherapy and in combination with other drugs.

However, within depression there exist various subindications, such as stress-mediated depression, wherein inflammatory markers are increased or abnormal.

Depressive patients having increased inflammatory markers or having abnormal levels of inflammatory markers compared to a reference value can in some circumstances be difficult to treat with some antidepressant compounds. One reason for this difficulty could be that treating these patients with an antidepressant compound is not normalizing the increased or abnormal levels of the inflammatory markers.

Therefore a need exists for a novel treatment of patients having a depression, such as stress-mediated depression, wherein in said patients the level of one or more inflammatory markers is increased or the level of one or more inflammatory markers is abnormal compared to a reference value.

It has now been found that inflammatory markers in animals are increased in a model of depression by exposing the animals to a stressful environment and that this increase in inflammatory markers is reversed by administration of gaboxadol to the stressed animals.

### SUMMARY OF THE INVENTION

In one aspect the present invention relates to a method for the treatment of depression comprising administering a therapeutically effective amount of gaboxadol to a patient, wherein the level of one or more inflammatory markers in said patient is increased or abnormal. In another aspect the present invention relates to a method for the treatment of stress-mediated depression comprising administering a therapeutically effective amount of gaboxadol to a patient, wherein the level of one or more inflammatory markers is increased or abnormal.

In another aspect the present invention relates to a method for the treatment of depression or the amelioration of one or more depressive symptoms comprising administering a therapeutically effective amount of gaboxadol to a patient, wherein the clinical presentation of one or more symptoms of depression are the physiological effect of a general medical condition.

In another aspect the present invention relates to a method for testing the therapeutic effectiveness of a compound in the treatment of depression or reducing the symptoms of depression comprising measuring the amount of one or more inflammatory markers in a sample from a patient before said compound is administered to the patient and comparing with the amount of said one or more inflammatory markers in a sample from the same patient after administration of said compound to the patient.

In another aspect the present invention relates to a method for the treatment of depression comprising the steps:
a. determining the amount of one or more inflammatory markers in a sample from a patient and comparing said amount with reference values of said one or more inflammatory markers;
b. administering of a therapeutically effective amount of gaboxadol to said patient if the amount of said one or more inflammatory markers is abnormal compared to said reference values.

In another aspect the present invention relates to use of gaboxadol for preparing a pharmaceutical composition for treating a patient having a depression, wherein the level of one or more inflammatory markers in said patient is increased or abnormal.

In another aspect the present invention relates to use of gaboxadol for preparing a pharmaceutical composition for treating a patient having stress-mediated depression, wherein the level of one or more inflammatory markers in said patient is increased or abnormal.

In another aspect the present invention relates to use of gaboxadol for preparing a pharmaceutical composition for treating a patient having a depression or the amelioration of one or more of a patient's depressive symptoms, wherein the clinical presentation of one or more symptoms of depression are the physiological effect of a general medical condition.

In another aspect the present invention relates to gaboxadol for use in a method for treating a patient having a depression, wherein the level of one or more inflammatory markers in said patient is increased or abnormal.

In another aspect the present invention relates to gaboxadol for use in a method for treating a patient having stress-mediated depression, wherein the level of one or more inflammatory markers is increased or abnormal in said patient.

In another aspect the present invention relates to gaboxadol for use in a method for treating a patient having a depression or the amelioration of one or more of a patient's depressive symptoms, wherein the clinical presentation of one or more symptoms of depression are the physiological effect of a general medical condition.

### DESCRIPTION OF THE INVENTION

In WO2004112786 gaboxadol has previously been showed to possess anti depressive actions in animal models. In a novel series of experiments we investigated the effects of gaboxadol on inflammatory markers in an animal model of depression. This animal model uses chronic mild unpredictive stress to induce an anhedonic state in the animals, which can be reversed by therapeutically active antidepressants. Surprisingly, gaboxadol dose dependently was able to reverse most of the chronic mild stress induced changes in cytokine levels, indicating a strong anti inflammatoric effect under stress related conditions. This effect only appears in animals with cytokine levels different from control animals and is therefore a consequence of the changes in the inflammatoric system and not a general effect. Gaboxadol is therefore able to specifically normalise inflammatory markers, such as cytokine levels in animals with abnormal cytokine levels. Since several stress related diseases have strong alterations in the cytokine levels as part of the underlying conditions, the present inventors propose that gaboxadol in these specific conditions will possess therapeutic advantages over current treatment, which generally does not affect the cytokine levels. Normalisation of cytokine levels is envisaged to predict increased responder rate, increased efficacy level and a reduced remission rate.

Definitions

The term “stress-mediated depression” sometimes also referred to as stress-induced depression, refers to depression as the result of a patient's past emotional experiences and/or physiological stress, such as medical illness or the medication associated with the medical illness, wherein one or more inflammatory markers is increased or abnormal.

The term “sample from a patient” refers to a biological sample from a patient and is intended to include tissues, cells, biological fluids, such as blood, and isolates thereof, isolated from a patient, as well as tissues, cells and fluids present within a patient.

As used herein, the term “patient” refers to any mammal. The patient, such as a human, to be treated with gaboxadol may in fact be any subject of the human population, male or female, which may be divided into children, adults, or elderly. Any one of these patient groups relates to an embodiment of the invention. In one embodiment, the subject is an elderly human. In one embodiment, the subject does not suffer from a sleep disorder or sleep condition.

As used herein, the term “therapeutically effective amount” refers to the amount/dose of a compound or pharmaceutical composition that is sufficient to produce an effective response (i.e., a biological or medical response of a tissue, system, animal or human sought by a researcher, veterinarian, medical doctor or other clinician) upon administration to a patient. The “therapeutically effective amount” will vary depending on inter alia the disease and its severity, and the age, weight, physical condition and responsiveness of the patient to be treated. Furthermore the “therapeutically effective amount” may vary if the compound of the invention is combined with one or more compounds, in such a case the amount of a given compound might be lower, such as a sub-effective amount.

As used herein, the term “treating” or “treatment” refers to preventing or delaying the appearance of clinical symptoms of a disease or condition in a patient that may be afflicted with or predisposed to the disease or condition, but does not yet experience or display clinical or subclinical symptoms of the disease or condition. “Treating” or “treatment” also refers to inhibiting the disease or condition, i.e., arresting or reducing its development or at least one clinical or subclinical symptom thereof. “Treating” or “treatment” further refers to relieving the disease or condition, i.e., causing regression of the disease or condition or at least one of its clinical or subclinical symptoms. The benefit to a patient to be treated is either statistically significant or at least perceptible to the patient and/or the physician. Nonetheless, prophylactic (preventive) and therapeutic (curative) treatment are two separate embodiments of the invention.

As used herein, the term “pharmaceutically acceptable” refers to molecular entities and compositions that are “generally regarded as safe”—e.g., that are physiologically tolerable and do not typically produce an allergic or similar untoward reaction, such as gastric upset and the like, when administered to a human. In another embodiment, this term refers to molecular entities and compositions approved by a regulatory agency of the federal or a state government or listed in the U.S. Pharmacopeia or another generally recognized pharmacopeia for use in animals, and more particularly in humans.

A physician can determine whether the level of one or more inflammatory markers is increased or is abnormal. This determination is based on the physician's sound judgment and can be based on comparison with reference values, such as from healthy individuals. In some circumstances it is the pattern in the level of the inflammatory markers that determines whether the level of the inflammatory markers is abnormal. In the physician's judgment are the following normally also taken into account, such as the relevant circumstances, including the condition to be treated, the age, weight, sex, genetic background and the race of the patient. In one embodiment of the invention an inflammatory marker is increased or abnormal if said inflammatory marker differs from a reference value, such as from healthy individuals, by more than 1%, or more than 5%, or more than 10%, or more than 15%, or more than 20%, or more than 25%, or more than 30%, or more than 35%, or more than 40%, or more than 45%, or more than 50%, or more than 55%, or more than 60%, or more than 65%, or more than 70%, or more than 75%, or more than 80%, or more than 85%, or more than 90%, or more than 95%, or more than 100%, or more than 150%.

The disorders that can be treated according to the present invention are known according to established and accepted classifications, which can be found in various sources. For example, at present, the fourth edition of the Diagnostic and Statistical Manual of Mental Disorders (DSM-IV™) (2000, American Psychiatric Association, Washington, D.C.), provides a diagnostic tool for identifying many of the disorders described herein. Also, the International Classification of Diseases, Tenth Revision, (ICD-10) provides classifications for many of the disorders described herein. The skilled person in the art will recognize that there are alternative nomenclatures, nosologies, and classification systems for disorders described herein, including those as described in the DMS-IV and ICD-10, and that terminology and classification systems evolve with medical scientific progress. Moreover the scientific literature also gives definition on disorders, for example the disorder burn-out is described in Rydmark et al, Biological Psychiatry, 2006, 60, 867-873.

Throughout this description, “gaboxadol” is intended to include any form of the compound, such as the free base (zwitter ion), pharmaceutically acceptable salts, e.g., pharmaceutically acceptable acid addition salts, hydrates or solvates of the base or salt, as well as anhydrates, and also amorphous, or crystalline forms.

In a further embodiment, gaboxadol is selected from the zwitter ion, typically a hydrate thereof, although the anhydrate is also suitable. A suitable embodiment is the zwitter ion monohydrate.

In a further embodiment, gaboxadol is selected from an acid addition salt, typically a pharmaceutically acceptable acid addition salt. A suitable embodiment is an organic acid addition salt, such as any one of the maleic, fumaric, benzoic, ascorbic, succinic, oxalic, bis-methylenesalicylic, methanesulfonic, ethane-disulfonic, acetic, propionic, tartaric, salicylic, citric, gluconic, lactic, malic, mandelic, cinnamic, citraconic, aspartic, stearic, palmitic, itaconic, glycolic, p-amino-benzoic, glutamic, benzene sulfonic or theophylline acetic acid addition salts, as well as the 8-halotheophyllines, for example 8-bromo-theophylline. Another suitable embodiment is an inorganic acid addition salt, such as any one of the hydrochloric, hydrobromic, sulfuric, sulfamic, phosphoric or nitric acid addition salts.

In another embodiment, gaboxadol is in the form of the hydrochloric acid salt, the hydrobromic acid salt, or the zwitter ion monohydrate.

In a further embodiment, gaboxadol is crystalline, such as the crystalline hydrochloric acid salt, the crystalline hydrobromic acid salt, or the crystalline zwitter ion monohydrate.

The acid addition salts according to the invention may be obtained by treatment of gaboxadol with the acid in an inert solvent followed by precipitation, isolation and optionally re-crystallization by known methods and if desired micronization of the crystalline product by wet or dry milling or another convenient process, or preparation of particles from a solvent-emulsification process. Suitable methods are described in EP Patent No. 0000338, for example.

Precipitation of the salt is typically carried out in an inert solvent, e.g., an inert polar solvent such as an alcohol (e.g., ethanol, 2-propanol and n-propanol), but water or mixtures of water and inert solvent may also be used.

In yet another embodiment of the present invention the inflammatory marker or the cytokine is selected from the group comprising Apo A1 (Apolipoprotein A1), Beta-2 Microglobulin, Clusterin, CRP (C Reactive Protein), Cystatin-C, Eotaxin, Factor VII, FGF-9 (Fibroblast Growth Factor-9), GCP-2 (Granulocyte Chemotactic Protein-2), Growth Hormone, IgA (Immunoglobulin A), IL-10 (Interleukin-10), IL-1beta (Interleukin-1beta), IL-2 (Interleukin-2), IL-4 (Interleukin-4), IL-5 (Interleukin-5), Insulin, IP-10 (Inducible Protein-10), Leptin, LIF (Leukemia Inhibitory Factor), MDC (Macrophage-Derived Chemokine), MIP-1alpha (Macrophage Inflammatory Protein-1alpha), MIP-1beta (Macrophage Inflammatory Protein-1beta), MIP-1gamma (Macrophage Inflammatory Protein-1gamma), MIP-2 (Macrophage Inflammatory Protein-2), MIP-3beta (Macrophage Inflammatory Protein-3beta), MPO (Myeloperoxidase), Myoglobin, NGAL (Lipocalin-2), OSM (Oncostatin M), Osteopontin, SAP (Serum Amyloid P), SCF (Stem Cell Factor), SGOT (Serum Glutamic-Oxaloacetic Transaminase), TIMP-1 (Tissue Inhibitor of Metalloproteinase Type-1), Tissue Factor, TPO (Thrombopoietin) and VEGF (Vascular Endothelial Cell Growth Factor).

In yet another embodiment of the present invention the therapeutically effective amount of gaboxadol ranges from 1 mg to 20 mg, such as 5 mg to 15 mg of gaboxadol per day.

In yet another embodiment of the present invention gaboxadol is administered as an oral dose form.

In yet another embodiment of the present invention gaboxadol is a solid oral dose form, such as tablets or capsules, or a liquid oral dose form.

In yet another embodiment of the present invention gaboxadol is crystalline.

In yet another embodiment of the present invention the patient is a human.

In yet another embodiment of the present invention the patient additionally is administered a therapeutically effective amount of escitalopram or a pharmaceutically acceptable salt thereof. In yet another embodiment the pharmaceutically acceptable salt of escitalopram is the oxalate salt, the HCl salt or the HBr salt of escitalopram.

In yet another embodiment of the present invention the level of one or more inflammatory markers is increased or abnormal in the patient to be treated.

In yet another embodiment of the present invention said stress-mediated depression is caused by work-related depression, burn-out, chronic fatigue syndrome, Post traumatic stress disorder (PTSD), exhaustion fatigue, exhaustion depression or acute stress disorder (ASD).

In yet another embodiment of the present invention said stress is caused by past emotional experiences, such as divorce, natural disaster, poverty, interpersonal conflicts, bereavement, coping with a medical illness or job loss.

In yet another embodiment of the present invention said stress is physiological stress, such as medical illness or the medication associated with the medical illness.

In yet another embodiment of the present invention the medical illness is multiple sclerosis, stroke, hypothyroidism, diabetes, such as type 1 or type 2, cardiac disease, such as acute myocardial infarct or angina pectoris, cancer, HIV infection or AIDS, a neurological disorder, such as cerobrovascular disorder, Parkinson's Disease, traumatic brain injury, stroke, such as left hemisphere stroke involving the dorsal lateral frontal cortex, chronic fatigue syndrome, fibromyalgia, neurocrine abnormalities, such as an increased secretion of cortisol and/or of corticotropin-releasing hormone, patients receiving cytokines, such as interferon-α and/or Interleukin-2, Post traumatic stress disorder (PTSD), burn-out, work-related depression, exhaustion fatigue, chronic pain conditions, dyslipidemia, dysthymia, an inflammatory disease, exhaustion depression or acute stress disorder (ASD).

In yet another embodiment of the present invention the general medical condition is multiple sclerosis, stroke, hypothyroidism, diabetes, such as type 1 or type 2, cardiac disease, such as acute myocardial infarct or angina pectoris, cancer, HIV infection or AIDS, a neurological disorder, such as cerobrovascular disorder, Parkinson's Disease, traumatic brain injury, stroke, such as left hemisphere stroke involving the dorsal lateral frontal cortex, chronic fatigue syndrome, fibromyalgia, neurocrine abnormalities, such as an increased secretion of cortisol and/or of corticotropin-releasing hormone, patients receiving cytokines, such as interferon-α and/or Interleukin-2, Post traumatic stress disorder (PTSD), burn-out, work-related depression, exhaustion fatigue, chronic pain conditions, dyslipidemia, dysthymia, an inflammatory disease, exhaustion depression or acute stress disorder (ASD).

In yet another embodiment of the present invention the compound to be tested for the therapeutic effectiveness is selected from antidepressant compounds, such as an SSRI, SNRI's, or other antidepressants compounds approved for such use by a government agency, such as the FDA. In yet another embodiment of the present invention this compound is gaboxadol.

In yet another embodiment of the present invention the pharmaceutical composition comprises from 1 mg to 20 mg, such as 5 mg to 15 mg of gaboxadol. In yet another embodiment of the present invention the pharmaceutical composition is as an oral dose form. In yet another embodiment of the present invention the pharmaceutical composition is a solid oral dose form, such as tablets or capsules, or a liquid oral dose form. In yet another embodiment of the present invention the pharmaceutical composition additionally comprises escitalopram or a pharmaceutically acceptable salt thereof. In yet another embodiment of the present invention the pharmaceutically acceptable salt of escitalopram is the oxalate salt, the HCl salt or the HBr salt of escitalopram.

In one embodiment of the present invention gaboxadol is for maintenance therapy.

Formulations

Gaboxadol may be administered as an oral dose form, such as a solid oral dose form, typically tablets or capsules, or as a liquid oral dose form. Gaboxadol may be administered in an immediate release dosage form or a controlled or sustained release dosage form. According to one embodiment, the dosage form provides controlled or sustained release of the gaboxadol in an amount less than a sleep-inducing amount. Gaboxadol may be conveniently administered orally in unit dosage forms, such as tablets or capsules, containing the active ingredient in an amount from about 0.1 to about 150 mg/day, from about 0.2 to about 100 mg/day, from about 0.5 to about 50 mg/day, from about 0.1 to about 50 mg/day, from about 1 to about 15 mg/day, or from about 2 to about 5 mg/day. Typically, the pharmaceutical composition comprises from about 0.5 mg to about 20 mg, such as about 0.5 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 10.5 mg, about 11 mg, about 11.5 mg, about 12 mg, about 12.5 mg, about 13 mg, about 13.5 mg, about 14 mg, about 14.5 mg, about 15 mg, about 15.5 mg, about 16 mg, about 16.5 mg, about 17 mg, about 17.5 mg, about 18 mg, about 18.5 mg, about 19 mg, about 19.5 mg or about 20 mg of gaboxadol. The amount of gaboxadol is calculated based on the free base (zwitter ion) form.

In one embodiment, gaboxadol is administered once daily (for example, in the morning or afternoon) using doses of about 1 mg to about 20 mg. In another embodiment, gaboxadol is administered in a more prolonged and continuous release using non-sleep-inducing concentrations of gaboxadol—e.g., administration 2-3 times daily with low doses or a modified release formulation prepared using conventional methods known in the art, such that about 5 to about 50 mg of gaboxadol are administered to the subject per 24 hour period. In yet another embodiment, gaboxadol is administered in an amount that is less than a sleep-inducing amount.

According to the present invention, gaboxadol or a pharmaceutically acceptable salt thereof may be administered in any suitable way, e.g., orally or parenterally, and it may be presented in any suitable form for such administration, e.g., in the form of tablets, capsules, powders, syrups or solutions or dispersions for injection. In another embodiment, and in accordance with the purpose of the present invention, gaboxadol is administered in the form of a solid pharmaceutical entity, suitably as a tablet or a capsule or in the form of a suspension, solution or dispersion for injection. Additionally, gaboxadol may be administered with a pharmaceutically acceptable carrier, such as an adjuvant and/or diluent.

Methods for the preparation of solid or liquid pharmaceutical preparations are well known in the art. See, e.g., Remington: The Science and Practice of Pharmacy, 21st ed., Lippincott Williams & Wilkins (2005). Tablets may thus be prepared by mixing the active ingredients with an ordinary carrier, such as an adjuvant and/or diluent, and subsequently compressing the mixture in a tabletting machine. Non-limiting examples of adjuvants and/or diluents include: corn starch, lactose, talcum, magnesium stearate, gelatine, lactose, gums, and the like. Any other adjuvant or additive such as colourings, aroma, and preservatives may also be used provided that they are compatible with the active ingredients. The pharmaceutical compositions of the invention thus typically comprise an effective amount of gaboxadol and a pharmaceutically acceptable carrier.

A suitable formulation of gaboxadol is described in WO 02/094225. Without limiting the invention in any way, it is intended that any one of the aspects or embodiments of this patent application is suitable for the medicaments or pharmaceutical compositions described herein. For example, WO 02/094225 entitled “Granular Preparations of Gaboxadol” relates to a specific melt granulation, which is particularly useful for formulation of an acid addition salt, but the present invention is in no way limited to such a formulation.

Pharmacological Tests

The following tests were performed to evaluate the potential effect of gaboxadol on stress related biochemical changes, using the Chronic mild stress model in rats (Jayatissa M N, Bisgaard C, Tingstrom A, Papp M, Wiborg O. Hippocampal cytogenesis correlates to escitalopram-mediated recovery in a chronic mild stress rat model of depression. Neuropsychopharmacology. November 2006; 31(11):2395-404). The biochemical changes induced in the animals studied in the stress models lead to increases in inflammatory mediators/markers.

At the end of the present study, terminal serum samples were collected for marker analysis to evaluate the effects of stress with and without drug on the levels of about 60 blood markers. Previously published data suggests that chronic mild stress modulates the expression of a number of blood plasma proteins. Differential modulation of this response by gaboxadol and escitalopram may be indicative of different mechanisms of action and may be alternative predictors of clinical efficacy.

Experimental Procedure

Animals

Serum samples were withdrawn from the tail, without stunning, during the daytime (09:00-17:00) 24 hrs after the last drug injection. Blood was collected into BD vacutainers containing clot activator and gel for serum preparation, inverted 5 times and maintained on ice until centrifugation at 3000 rpm for 10 min at 4° C. Serum was decanted, placed on ice, and at the end of the day stored at −80° C. Animal treatment groups were as follows (see table 1):

TABLE 1
Animal sets.
ANIMAL SETS

| | | Drug Treatment |
|---|---|---|
| Treatment Code | Stress Conditioning | or Behavior |
| CMS-VEH | Chronic Mild Stress | Vehicle |
| CMS-ECT | Chronic Mild Stress | Escitalopram |
| CMS-GBX-5 mg/kg | Chronic Mild Stress | Gaboxadol |
| CMS-GBX-10 mg/kg | Chronic Mild Stress | Gaboxadol |
| CMS-RES | Chronic Mild Stress | Resistant |
| NS-VEH | Non-Stressed | Vehicle |
| NS-ECT | Non-Stressed | Escitalopram |
| NS-GBX | Non-Stressed | Gaboxadol |

Analysis

Subsequent analyses of Serum samples showed the following (see table 2):

TABLE 2
Median values of each tested factor for each animal treatment group.

| | | median values | | | | | | |
|---|---|---|---|---|---|---|---|---|
| | | | | | | 2nd | | |
| | | 1st study | | | | study | | |
| | | Blood parameter | | | | | | |
| | | | | GBX | | | | |
| | | control | stress | 5 mg/kg | ESC 5 mg/kg | control | stress | GBX 10 mg/kg |
| Apo A1 (Apolipoprotein | ug/mL | 7.00 | 7.30 | 8.20 | 8.40 | 4.80 | 5.80 | 6.20 |
| A1) | | | | | | | | |
| Beta-2 Microglobulin | ug/mL | | | | | 45.00 | 59.00 | 38.00 |
| Calbindin | ng/mL | | | | | 0.40 | 0.12 | |
| Clusterin | ug/mL | | | | | 110.00 | 146.00 | 108.00 |
| CRP (C Reactive Protein) | ug/mL | 1235.00 | 1105.00 | 1140.00 | 1060.00 | 1130.00 | 1190.00 | 1030.00 |
| Cystatin-C | ng/mL | 1045.00 | 1440.00 | 1165.00 | | 717.00 | 956.00 | 696.00 |
| Eotaxin | pg/mL | 851.00 | 1210.00 | | 1300.00 | 1362.00 | 1656.00 | 1072.00 |
| Factor VII | ng/mL | | | | | 0.48 | 0.07 | 0.30 |
| FGF-9 (Fibroblast Growth | ng/mL | 0.38 | 0.95 | 0.59 | 0.90 | 1.70 | 2.40 | 1.70 |
| Factor-9) | | | | | | | | |
| GCP-2 (Granulocyte | ng/mL | 0.14 | 0.23 | 0.19 | 0.21 | 0.28 | 0.31 | 0.29 |
| Chemotactic Protein-2) | | | | | | | | |
| Growth Hormone | ng/mL | 1.66 | 5.67 | 7.67 | 1.38 | 12.00 | 23.00 | 3.10 |
| GST-alpha (Glutathione | ng/mL | | | | | 0.89 | 0.40 | |
| S-Transferase alpha) | | | | | | | | |
| GST-Mu | ng/mL | | | | | 2080.00 | 60.00 | 1190.00 |
| IgA (Immunoglobulin A) | ug/mL | 7.10 | 6.83 | 5.91 | 6.54 | 5.70 | 7.60 | 4.30 |
| IL-10 (Interleukin-10) | pg/mL | 278.00 | 228.00 | 249.00 | 317.00 | 360.00 | 411.00 | 362.00 |
| IL-11 (Interleukin-11) | pg/mL | 129.00 | 105.00 | 128.00 | 166.00 | 164.00 | 98.00 | |
| IL-18 (Interleukin-18) | ng/mL | 0.07 | 0.14 | 0.07 | | 0.53 | 0.85 | 0.43 |
| IL-1alpha (Interleukin- | pg/mL | 8.12 | 13.80 | 11.30 | 15.40 | | | |
| 1alpha) | | | | | | | | |
| IL-1beta (Interleukin- | ng/mL | | | | | 0.53 | 0.65 | 0.56 |
| 1beta) | | | | | | | | |
| IL-2 (Interleukin-2) | pg/mL | 22.30 | 38.80 | 31.30 | 38.80 | 33.00 | 46.00 | 22.00 |
| IL-4 (Interleukin-4) | pg/mL | 24.65 | 46.70 | 29.60 | 51.70 | | | |
| IL-5 (Interleukin-5) | ng/mL | | | | | 0.26 | 0.51 | 0.24 |
| IL-7 (Interleukin-7) | ng/mL | 0.11 | 0.22 | 0.16 | 0.22 | 0.04 | 0.04 | 0.06 |
| Insulin | uIU/mL | 10.90 | 12.20 | 8.88 | 11.30 | 9.40 | 3.10 | 14.50 |
| IP-10 (Inducible Protein- | pg/mL | 86.70 | 186.00 | 138.00 | 194.00 | 31.00 | 36.00 | 41.00 |
| 10) | | | | | | | | |
| Leptin | ng/mL | 3.24 | 3.17 | 2.59 | 2.62 | 0.94 | 0.27 | 1.35 |
| LIF (Leukemia Inhibitory | pg/mL | 36.60 | 70.50 | 53.90 | 67.20 | 49.00 | 93.00 | 67.00 |
| Factor) | | | | | | | | |
| Lymphotactin | pg/mL | 41.80 | 73.70 | 53.80 | 68.00 | 64.00 | 72.00 | 63.00 |
| MCP-1 (Monocyte | pg/mL | 1095.00 | 1095.00 | 1185.00 | 1500.00 | 1065.00 | 1025.00 | 1020.00 |
| Chemoattractant Protein- | | | | | | | | |
| 1) | | | | | | | | |
| MCP-3 (Monocyte | pg/mL | 566.00 | 657.00 | 655.00 | 885.00 | 653.00 | 703.00 | 659.00 |
| Chemoattractant Protein- | | | | | | | | |
| 3) | | | | | | | | |
| MCP-5 (Monocyte | pg/mL | 0.76 | 0.73 | 0.77 | 0.87 | 0.75 | 0.75 | 0.72 |
| Chemoattractant Protein- | | | | | | | | |
| 5) | | | | | | | | |
| M-CSF (Macrophage- | ng/mL | 813.00 | 875.00 | 865.00 | 941.00 | 1960.00 | 1610.00 | 1400.00 |
| Colony Stimulating | | | | | | | | |
| Factor) | | | | | | | | |
| MDC (Macrophage- | pg/mL | | | | | 0.13 | 0.26 | 0.12 |
| Derived Chemokine) | | | | | | | | |
| MIP-1alpha (Macrophage | ng/mL | 116.00 | 209.00 | 173.00 | 244.00 | 120.00 | 107.00 | 127.00 |
| Inflammatory Protein- | | | | | | | | |
| 1alpha) | | | | | | | | |
| MIP-1beta (Macrophage | pg/mL | | | | | 0.01 | 0.02 | 0.01 |
| Inflammatory Protein- | | | | | | | | |
| 1beta) | | | | | | | | |
| MIP-1gamma | ng/mL | 6.42 | 13.10 | 7.95 | 11.50 | 16.00 | 15.00 | 19.00 |
| (Macrophage | | | | | | | | |
| Inflammatory Protein- | | | | | | | | |
| 1gamma) | | | | | | | | |
| MIP-2 (Macrophage | pg/mL | 0.05 | 0.11 | 0.08 | 0.09 | 0.21 | 0.34 | 0.22 |
| Inflammatory Protein-2) | | | | | | | | |
| MIP-3beta (Macrophage | ng/mL | | | | | 21.75 | 13.70 | 29.70 |
| Inflammatory Protein- | | | | | | | | |
| 3beta) | | | | | | | | |
| MPO (Myeloperoxidase) | ng/mL | 89.40 | 53.80 | 20.80 | 54.60 | 610.00 | 39.85 | 533.00 |
| Myoglobin | ng/mL | | | | | 408.00 | 351.00 | 520.00 |
| NGAL (Lipocalin-2) | ng/mL | 0.21 | 0.12 | 0.17 | 0.22 | 0.10 | 0.11 | 0.15 |
| OSM (Oncostatin M) | ng/mL | | | | | 21.95 | 33.90 | 19.10 |
| Osteopontin | ng/mL | 188.00 | 391.00 | 396.00 | 310.00 | 356.00 | 509.00 | 255.00 |
| RANTES (Regulation | pg/mL | | | | | 12.85 | 11.25 | 10.60 |
| Upon Activation, Normal | | | | | | | | |
| T-Cell Expressed and | | | | | | | | |
| Secreted) | | | | | | | | |
| SAP (Serum Amyloid P) | ug/mL | 135.00 | 173.00 | 133.00 | 178.00 | 241.00 | 198.00 | 403.00 |
| SCF (Stem Cell Factor) | pg/mL | 12.55 | 15.30 | 14.85 | 16.95 | 39.20 | 50.80 | 11.30 |
| SGOT (Serum Glutamic- | ug/mL | 0.16 | 0.27 | 0.21 | 0.31 | 6.16 | 6.14 | 8.53 |
| Oxaloacetic | | | | | | | | |
| Transaminase) | | | | | | | | |
| TIMP-1 (Tissue Inhibitor | ng/mL | 0.09 | 0.28 | 0.16 | 0.22 | 0.37 | 0.77 | 0.24 |
| of Metalloproteinase | | | | | | | | |
| Type-1) | | | | | | | | |
| Tissue Factor | ng/mL | 0.08 | 0.13 | 0.10 | 0.15 | 0.06 | 0.03 | 0.06 |
| TNF-alpha (Tumor | ng/mL | | | | | 2.55 | 4.14 | 4.45 |
| Necrosis Factor-alpha) | | | | | | | | |
| TPO (Thrombopoietin) | ng/mL | 134.00 | 142.00 | 147.00 | 142.00 | 130.00 | 167.00 | 119.00 |
| VCAM-1 (Vascular Cell | ng/mL | 299.00 | 381.00 | 361.00 | 409.00 | 330.00 | 311.00 | 353.00 |
| Adhesion Molecule-1) | | | | | | | | |
| VEGF (Vascular | pg/mL | 55.00 | 89.00 | 81.00 | 106.00 | 69.00 | 243.00 | 78.00 |
| Endothelial Cell Growth | | | | | | | | |
| Factor) | | | | | | | | |
| vWF (von Willebrand | ng/mL | | | | | | | |
| Factor) | | | | | | | | |

Data

Numerical values of the concentration of each factor for each animal were collected.

Results and conclusion

The above-described pharmacological testing showed that chronic mild stress significantly alters a number of serum protein markers compared to non-stressed controls. Rats that did not show a behavioral response following CMS treatment (CMS-RES group) did not show a significant upregulation of these serum protein markers compared to non-stressed controls. The present inventors found that treatment with gaboxadol significantly reversed the stress-induced alterations in serum markers toward levels found in non-stressed controls.

Thus, from the foregoing testing and results, the inventors discovered that chronic mild stress significantly alters the expression of a set of serum marker proteins, and that partial or full reversal of this effect with gaboxadol (but not with escitalopram) suggests that gaboxadol affects stress-related biochemical changes by reducing inflammatory mediators. Thus gaboxadol, dose dependently is able to reverse or partially reverse changes at most in inflammatory parameters induced by chronic mild stress. In contrast, escitalopram was inactive at most of these changes. Thus, gaboxadol can be used to effectively treat stress-mediated depression wherein one or more of these inflammatory parameters are implicated in the course of the progression of depression.

All non-patent references, patents, and patent applications cited and discussed in this specification are incorporated herein by reference in their entirety and to the same extent as if each was individually incorporated by reference.

1. A method for the treatment of depression comprising administering a therapeutically effective amount of gaboxadol to a patient, wherein the level of one or more inflammatory markers in said patient is increased or abnormal.

2. The method of claim 1, wherein said gaboxadol is administered as maintenance therapy.

3. The method of claim 1, wherein said one or more inflammatory markers are selected from the group consisting of Apo A1 (Apolipoprotein A1), Beta-2 Microglobulin, Clusterin, CRP (C Reactive Protein), Cystatin-C, Eotaxin, Factor VII, FGF-9 (Fibroblast Growth Factor-9), GCP-2 (Granulocyte Chemotactic Protein-2), Growth Hormone, IgA (Immunoglobulin A), IL-10 (Interleukin-10), IL-1beta (Interleukin-1beta), IL-2 (Interleukin-2), IL-4 (Interleukin-4), IL-5 (Interleukin-5), Insulin, IP-10 (Inducible Protein-10), Leptin, LIF (Leukemia Inhibitory Factor), MDC (Macrophage-Derived Chemokine), MIP-1alpha (Macrophage Inflammatory Protein-1alpha), MIP-1beta (Macrophage Inflammatory Protein-1beta), MIP-1gamma (Macrophage Inflammatory Protein-1gamma), MIP-2 (Macrophage Inflammatory Protein-2), MIP-3beta (Macrophage Inflammatory Protein-3beta), MPO (Myeloperoxidase), Myoglobin, NGAL (Lipocalin-2), OSM (Oncostatin M), Osteopontin, SAP (Serum Amyloid P), SCF (Stem Cell Factor), SGOT (Serum Glutamic-Oxaloacetic Transaminase), TIMP-1 (Tissue Inhibitor of Metalloproteinase Type-1), Tissue Factor, TPO (Thrombopoietin), and VEGF (Vascular Endothelial Cell Growth Factor).

4. The method of claim 1, wherein gaboxadol is in the form of an acid addition salt, or a zwitterion hydrate or zwitterion anhydrate.

5. The method of claim 1, wherein gaboxadol is in the form of a pharmaceutically acceptable acid addition salt selected from the group consisting of hydrochloride and hydrobromide salts, or in the form of a zwitterion monohydrate.

6. The method of claim 1, wherein the therapeutically effective amount ranges from 1 mg to 20 mg of gaboxadol per day.

7. The method of claim 1, wherein gaboxadol is administered as an oral dose form.

8. The method of claim 1, wherein gaboxadol is a solid oral dose form, or a liquid oral dose form.

9. The method of claim 1, wherein said gaboxadol is crystalline.

10. The method of claim 1, wherein said patient is a human.

11. The method of claim 1, wherein the patient additionally is administered a therapeutically effective amount of escitalopram or a pharmaceutically acceptable salt thereof.

12. The method of claim 11 wherein the pharmaceutically acceptable salt of escitalopram is the oxalate salt, the HCl salt or the HBr salt of escitalopram.

13. A method for the treatment of stress-mediated depression comprising administering a therapeutically effective amount of gaboxadol to a patient, wherein the level of one or more inflammatory markers is increased or abnormal in said patient.

14. The method of claim 13, wherein said gaboxadol is administered as maintenance therapy.

15. The method of claim 13, wherein said stress-meditated depression is caused by work-related depression, burn-out, chronic fatigue syndrome, Post traumatic stress disorder (PTSD), exhaustion fatigue, exhaustion depressions, or acute stress disorder (ASD).

16. The method of claim 13, wherein said stress is caused by past emotional experiences.

17. The method of claim 13, wherein said stress is physiological stress.

18. The method of claim 17, wherein the medical illness is selected from the group consisting of multiple sclerosis, stroke, hypothyroidism, diabetes, cardiac disease, cancer, HIV infection or AIDS, a neurological disorder, Parkinson's Disease, traumatic brain injury, stroke, chronic fatigue syndrome, fibromyalgia, neurocrine abnormalities illness associated with administration of cytokines, Post traumatic stress disorder (PTSD), burn-out, work-related depression, exhaustion fatigue, chronic pain conditions, dyslipidemia, dysthymia, an inflammatory disease, exhaustion depression, and acute stress disorder (ASD).

19. The method of claim 13, wherein gaboxadol is in the form of an acid addition salt, or a zwitterion hydrate or zwitterion anhydrate.

20. The method of claim 13, wherein gaboxadol is in the form of a pharmaceutically acceptable acid addition salt selected from the group consisting of hydrochloride and hydrobromide salts, or in the form of a zwitterion monohydrate.

21. The method of claim 13, wherein the therapeutically effective amount ranges from 1 mg to 20 mg of gaboxadol per day.

22. The method of claim 13, wherein gaboxadol is administered as an oral dose form.

23. The method of claim 13, wherein gaboxadol is a solid oral dose form, or a liquid oral dose form.

24. The method of claim 13, wherein said gaboxadol is crystalline.

25. The method of claim 13, wherein said patient is a human.

26. The method of claim 13, wherein the patient additionally is administered a therapeutically effective amount of escitalopram or a pharmaceutically acceptable salt thereof.

27. The method of claim 26 wherein the pharmaceutically acceptable salt of escitalopram is the oxalate salt, the HCl salt or the HBr salt of escitalopram.

28. The method of claim 13, wherein the inflammatory marker is selected from the group consisting of Apo A1 (Apolipoprotein A1), Beta-2 Microglobulin, Clusterin, CRP (C Reactive Protein), Cystatin-C, Eotaxin, Factor VII, FGF-9 (Fibroblast Growth Factor-9), GCP-2 (Granulocyte Chemotactic Protein-2), Growth Hormone, IgA (Immunoglobulin A), IL-10 (Interleukin-10), IL-1beta (Interleukin-1beta), IL-2 (Interleukin-2), IL-4 (Interleukin-4), IL-5 (Interleukin-5), Insulin, IP-10 (Inducible Protein-10), Leptin, LIF (Leukemia Inhibitory Factor), MDC (Macrophage-Derived Chemokine), MIP-1alpha (Macrophage Inflammatory Protein-1alpha), MIP-1beta (Macrophage Inflammatory Protein-1beta), MIP-1gamma (Macrophage Inflammatory Protein-1gamma), MIP-2 (Macrophage Inflammatory Protein-2), MIP-3beta (Macrophage Inflammatory Protein-3beta), MPO (Myeloperoxidase), Myoglobin, NGAL (Lipocalin-2), OSM (Oncostatin M), Osteopontin, SAP (Serum Amyloid P), SCF (Stem Cell Factor), SGOT (Serum Glutamic-Oxaloacetic Transaminase), TIMP-1 (Tissue Inhibitor of Metalloproteinase Type-1), Tissue Factor, TPO (Thrombopoietin), and VEGF (Vascular Endothelial Cell Growth Factor).

29. A method for the treatment of depression or the amelioration of one or more depressive symptoms comprising administering a therapeutically effective amount of gaboxadol to a patient, wherein the clinical presentation of one or more symptoms of depression are the physiological effect of a general medical condition.

30. The method of claim 29, wherein the general medical condition is selected from the group consisting of: multiple sclerosis, stroke, hypothyroidism, diabetes, cardiac disease, cancer, HIV infection or AIDS, a neurological disorder, Parkinson's Disease, traumatic brain injury, stroke, chronic fatigue syndrome, fibromyalgia, neurocrine abnormalities, illness associated with administration of cytokines, Post traumatic stress disorder (PTSD), burn-out, work-related depression, exhaustion fatigue, chronic pain conditions, dyslipidemia, dysthymia, an inflammatory disease, exhaustion depression, and acute stress disorder (ASD).

31. The method of claim 29 wherein gaboxadol is in the form of an acid addition salt, or a zwitterion hydrate or zwitterion anhydrate.

32. The method of claim 29, wherein gaboxadol is in the form of a pharmaceutically acceptable acid addition salt selected from the group consisting of hydrochloride and hydrobromide salts, or in the form of a zwitterion monohydrate.

33. The method of claim 29, wherein the therapeutically effective amount ranges from 1 mg to 20 mg of gaboxadol per day.

34. The method of claim 29, wherein gaboxadol is administered as an oral dose form.

35. The method of claim 29, wherein gaboxadol is a solid oral dose form, or a liquid oral dose form.

36. The method of claim 29, wherein said gaboxadol is crystalline.

37. The method of claim 29, wherein said patient is a human.

38. The method of claim 29, wherein the patient additionally is administered a therapeutically effective amount of escitalopram or a pharmaceutically acceptable salt thereof.

39. The method of claim 38 wherein the pharmaceutically acceptable salt of escitalopram is the oxalate salt, the HCl salt or the HBr salt of escitalopram.

40. A method for testing the therapeutic effectiveness of a compound in the treatment of depression or reducing the symptoms of depression comprising measuring the amount of one or more inflammatory markers in a sample from a patient before said compound is administered to the patient and comparing with the amount of said one or more inflammatory markers in a sample from the same patient after administration of said compound to the patient.

41. The method of claim 40, wherein the compound is selected from antidepressant compounds.

42. The method of claim 40 wherein the compound is gaboxadol.

43. The method of claim 40, wherein the inflammatory marker is selected from the group consisting of Apo A1 (Apolipoprotein A1), Beta-2 Microglobulin, Clusterin, CRP (C Reactive Protein), Cystatin-C, Eotaxin, Factor VII, FGF-9 (Fibroblast Growth Factor-9), GCP-2 (Granulocyte Chemotactic Protein-2), Growth Hormone, IgA (Immunoglobulin A), IL-10 (Interleukin-10), IL-1beta (Interleukin-1beta), IL-2 (Interleukin-2), IL-4 (Interleukin-4), IL-5 (Interleukin-5), Insulin, IP-10 (Inducible Protein-10), Leptin, LIF (Leukemia Inhibitory Factor), MDC (Macrophage-Derived Chemokine), MIP-1alpha (Macrophage Inflammatory Protein-1alpha), MIP-1beta (Macrophage Inflammatory Protein-1beta), MIP-1gamma (Macrophage Inflammatory Protein-1gamma), MIP-2 (Macrophage Inflammatory Protein-2), MIP-3beta (Macrophage Inflammatory Protein-3beta), MPO (Myeloperoxidase), Myoglobin, NGAL (Lipocalin-2), OSM (Oncostatin M), Osteopontin, SAP (Serum Amyloid P), SCF (Stem Cell Factor), SGOT (Serum Glutamic-Oxaloacetic Transaminase), TIMP-1 (Tissue Inhibitor of Metalloproteinase Type-1), Tissue Factor, TPO (Thrombopoietin), and VEGF (Vascular Endothelial Cell Growth Factor).

44. A method for the treatment of depression comprising the steps:
a. determining the amount of one or more inflammatory markers in a sample from a patient and comparing said amount with reference values of said one or more inflammatory markers;
b. administering of a therapeutically effective amount of gaboxadol to said patient if the amount of said one or more inflammatory markers is abnormal compared to said reference values.

45. The method of claim 44, wherein the inflammatory marker is selected from the group consisting of Apo A1 (Apolipoprotein A1), Beta-2 Microglobulin, Clusterin, CRP (C Reactive Protein), Cystatin-C, Eotaxin, Factor VII, FGF-9 (Fibroblast Growth Factor-9), GCP-2 (Granulocyte Chemotactic Protein-2), Growth Hormone, IgA (Immunoglobulin A), IL-10 (Interleukin-10), IL-1beta (Interleukin-1beta), IL-2 (Interleukin-2), IL-4 (Interleukin-4), IL-5 (Interleukin-5), Insulin, IP-10 (Inducible Protein-10), Leptin, LIF (Leukemia Inhibitory Factor), MDC (Macrophage-Derived Chemokine), MIP-1alpha (Macrophage Inflammatory Protein-1alpha), MIP-1 beta (Macrophage Inflammatory Protein-1beta), MIP-1 gamma (Macrophage Inflammatory Protein-1gamma), MIP-2 (Macrophage Inflammatory Protein-2), MIP-3beta (Macrophage Inflammatory Protein-3beta), MPO (Myeloperoxidase), Myoglobin, NGAL (Lipocalin-2), OSM (Oncostatin M), Osteopontin, SAP (Serum Amyloid P), SCF (Stem Cell Factor), SGOT (Serum Glutamic-Oxaloacetic Transaminase), TIMP-1 (Tissue Inhibitor of Metalloproteinase Type-1), Tissue Factor, TPO (Thrombopoietin), and VEGF (Vascular Endothelial Cell Growth Factor).

46. The method of claim 44, wherein gaboxadol is in the form of an acid addition salt, or a zwitterion hydrate or zwitterion anhydrate.

47. The method of claim 44, wherein gaboxadol is in the form of a pharmaceutically acceptable acid addition salt selected from the group consisting of hydrochloride of and hydrobromide salts, or in the form of the a zwitterion monohydrate.

48. The method of claim 44, wherein the therapeutically effective amount ranges from 1 mg to 20 mg of gaboxadol per day.

49. The method of claim 44, wherein gaboxadol is administered as an oral dose form.

50. The method of claim 44, wherein gaboxadol is a solid oral dose form, or a liquid oral dose form.

51. The method of claim 44, wherein said gaboxadol is crystalline.

52. The method of claim 44, wherein said patient is a human.

53. The method of claim 44, wherein the patient additionally is administered a therapeutically effective amount of escitalopram or a pharmaceutically acceptable salt thereof.

54. The method of claim 53 wherein the pharmaceutically acceptable salt of escitalopram is the oxalate salt, the HCl salt or the HBr salt of escitalopram.

55-132. (canceled)
